(19) United States
(12) Patent Application Publication
(10) Pub. No.: US 2014/0277231 A1
(43) Pub. Date: Sep. 18, 2014

(54) MEDICAL DEVICE INVOLVING MUSCLE STIMULATION ELECTRODES AND AN ELECTROMAGNETIC LEAD

(71) Applicant: Laurent Berneman, Paris (FR)

(72) Inventor: Laurent Berneman, Paris (FR)

(21) Appl. No.: 14/360,130

(22) PCT Filed: Nov. 24, 2011
(86) PCT No.: PCT/FR2011/000622

§ 371 Date: May 22, 2014

(30) Foreign Application Priority Data
Nov. 24, 2011 (FR) 1103598

(51) Int. Cl. A61N 1/05; A61N 1/362

(52) U.S. Cl. CPC A61N 1/05; A61N 1/362
USPC 607/11; 607/116; 607/60

(57) ABSTRACT

The invention relates to electrodes of a novel type in a heart stimulator functioning on energy produced by a piezoelectric effect. A variant of the electrodes is formed by the presence of a ferromagnetic material. The device constituting the heart stimulator has a small overall volume, weighs less than traditional stimulators, poses less risk of infection and clotting and much less risk of mechanical failure. Use of the device containing electrodes composed of materials capable of producing a piezoelectric effect and, if appropriate, also containing a ferromagnetic material, and also a directional probe comprising an electromagnet.

The invention relates to the field of muscle stimulators and in particular to cardiac stimulators (also known as pacemakers).

The invention relates to a muscle stimulation device involving at least one electrode according to the invention which operates by piezoelectric effect and of which one variant (the variant electrode) is made, in addition to the piezoelectric component part, of a ferromagnetic component part.

The medical device according to the invention includes on the one hand a variant electrode, as described above, and on the other hand, at least one lead containing an electromagnetic element, associated to the variant electrode.

The medical applications of the present invention aim at simplifying the working process of a new cardiac stimulator, making easier its implantation and its explantation by using the variant electrode and the associated steerable lead [symbol] S [symbol] . The variant device according to the invention is constituted in particular partly of a variant electrode with a ferromagnetic element and an operator-directed steerable lead [symbol] S [symbol] and including an electromagnet on its surface or within its constituting material. The working process of the medical device involves an energy source originated from the piezoelectric effect allowing the new cardiac stimulator to work. The lead [symbol] S [symbol] has its own energy source which enables the activation of an electromagnet when requested, creating a magnetic field close to the targeted electrode. The transmission of electrical energy, which is distinct from the electrode's energy, to a precise place of the human body is controlled by a steerable lead S powered by the said electrical current, inducing a short range electromagnetic effect.

The present invention relates to a new device comprising in particular a steerable lead commonly used in electrophysiology and as a variant containing an electromagnet. The device includes also an electrode which can autonomously produce and transmit an electrical signal, without any supply from an external energy source, to a tissue or an organ such as a tissue constituted of human or animal myocardic cells.

This electrode is also able to detect electrical activity in its vicinity. A variant electrode is characterized by the presence of a ferromagnetic material in addition to the other elements of the electrode according to the invention.

This electrode is connected to a second element (box) using a connecting lead which is distinct from the steerable lead [symbol] S [symbol] . The lead can be made of an electrical wire coated with an electrical insulating and hypoallergenic material. The electrode can also be connected to the box by a wireless communication way (with WiFi for example). The electrode, according to the invention, is partly constituted of a ferromagnetic material made of a metal with ferromagnetic properties as well as materials with piezoelectric properties. The said electrode can be covered with alternated thin metallic layers.

Cardiac pacemakers are usually made of two parts connected to each other by elements involving electrical wires. Those 2 parts comprise notably a case for one part and for the other part-working electrodes, that is they emit locally an electrical current where they are implanted in myocardic tissue; the whole device allows to fix a defined contraction frequency.

Experimentally and despite the great care used by surgeons or cardiologists or rythmologists, infectious endocarditis (that is to say, the infection of the internal lining of the heart) are common and burdened with a high mortality due mainly to the leads containing the electrodes at their end. This forces doctors to remove both parts of the pacemaker (case and electrodes and consequently the connecting leads) to ensure aseptic wounds for the patient and to install a new pacemaker if the need for stimulation is necessary. The cost of such operations is high for hospitals and none of the present devices can offer a way to reduce costs and surgical risks.

The devices of the prior art are formed with 3 distinct functional blocks comprising an energy generator (for example batteries), powering an electrical signals transmitter/receiver—which continues to the electrode. The generator supplies the electronic brain and the internal memory. The electronic brain is the whole of programmed paradigms to define a suitable stimulation response related to data collected by the electrode. Electronic brain also defines which elements are to be kept in the internal memory, again according to its own program. For example the state of the art is represented by cardiac devices and pacemakers called “classics” from that of the present invention.

We can refer to those described in the following patent applications and patents U.S.: U.S. Pat. No. 5,411,535, 2007/088398, U.S. Pat. No. 7,650,186 or No. 2007/02 19 590 or No. 2009/0082827 or No. 2009/0204170.

One can also refer to those marketed by Medtronic, Sorin Group, Boston Scientific, St Jude Medical, Biotronik that are all using direct connection (notably as electrical wires) between the electrodes and the case which is the only supplier of energy.

The exact localization of the electrode in the patient is difficult and requires to perform an operation on the heart muscle to remove the electrode.

The present invention provides a new design of a device whose aims among others are to reduce the risks of infection and dysfunction associated with permanent means of connection between the electrodes and the case which is the only provider of energy and secondly to implant cases with a smaller size (since the energy contained within the control box serves only to its operations and not to the entire device box/lead and electrode). It also relates to a device comprising, in addition to the electrode, a new steerable lead “S” containing at its end or near its end on or in its walls, an electromagnet.

Moreover, the present invention provides a longer service life compared with existing devices. This is allowed by the active participation of the electrode to produce energy for the system and the easy extraction and replacement of the electrode by using an electrode having a hollow portion including in particular a ferromagnetic element. When extraction of the electrode is needed, it is easier to reach it because it has been disengaged from the overlying amorphous tissue; the said electrode comprising a piezoelectric material and also a ferromagnetic element, and an extraction/implantation rod. The rod of said electrode is positioned in the interior cavity of the electrode. The latter has been moved thanks to the electromagnetic effect resulting from the application of a magnetic field by the steerable lead “S” close to the ferromagnetic element of said electrode. The entry of the extraction rod into the recess of the electrode will be interdependent on the electrode by a reverse movement made at the implantation and will allow to retract element 18 of FIG. 3.

The invention which will be described in detail below also includes variants with the same features and benefits.

The invention relates to a new device or system comprising two separate elements not interconnected by electrical wires or the equivalent and constituted by a box for the first and the second by electrodes; the whole system is a new type of pacemaker.

The invention relates to a device consisting of new electrodes, the new use of these electrodes containing a ferromagnetic element and having an internal source of energy produced by a piezoelectric effect upon implantation or attachment in the myocardium or skeletal muscle and a steerable lead “S”. An electrode according to the invention contains totally or partly one or more materials capable, under the effect of implantation in the myocardium or the skeletal muscle, of producing energy by a piezoelectric effect, to power all or part of the electrode's functions:
Electrode's working (in particular, to create an electrical pulse in a controlled manner in relation to time, intensity and amplitude) leading to depolarization of the myocardium,
The collection of in situ electrical potentials and
Communication with the box if needed and without excluding other possibilities of use.

Producing piezoelectricity in the electrode according to the invention can be obtained with a physical stress felt by the electrode (as an illustration of the invention), for example with the increase of the blood pressure in the heart's cavity or with the kinetic energy when the patient moves or breathes.

The same effect could be achieved with a myocardium which would contain fœtal cells or iPS cells built in 2006 by Yamanaka and colleagues and published in Cell, for example, 2007b, Volume 131, pages 861-872 (IPS or induced pluripotent cells designated) or differentiated or in the course of differentiation as myocardial cells.

The piezoelectric effect has been demonstrated for more than a century. The piezoelectric effect was produced with objects such as ceramic discs associated with metal elements by Lebrun, Gaulin et al. and published in 1996 in the Journal of electricity and electronics (Volume 9 pages 49-55) but this article does not describe nor suggest the preparation of electrodes or any use of this type of object coated with a material having piezoelectric effects, to produce a cardiac stimulating device or striated muscles stimulator.

General informations on the piezoelectric effect are available on the website Wikipedia.org.

The piezoelectric effect is generated from a known physical mechanism that requires the presence of materials such as metals or metal derivated (The ferro-electric materials of the perovskite structure, the LZT (ie, Lead Zirconate Titanate)) or barium titanate BaTiO3 or potassium niobate KNbO 3 or MEMS (ie micro electromechanical systems) or the semiconductor of III-V group of zinc-blende structure and II-VI wurtzite structure, quartz etc . . . ).

In the present invention, the electrode implanted in the myocardium is covered directly or indirectly, entirely or partly, with a material with piezo-electric effect which will produce an electric current due to an external physical stress exerted on the metal contained in or on the electrode or exerted on the combination or on the material containing said metal in which it is composed. Physical stress is for example increased blood pressure in the heart that occurs physiologically.

Materials which produce such an effect are known by experts in the field. As an illustration, the following materials are suitable to be incorporated in the electrode according to the invention unless they induce or cause allergic signs in a patient: Ferroelectric materials with perovskite structure, LZT (ie lead zirconate titanate) and its derivatives or MEMS (ie microelectromechanical system) or semiconductors of group III-V of zinc-blende structure and II-VI wurtzite structure, quartz etc . . .

The most suitable material will be determined by performing simple tests already validated in cosmetology or the construction of orthopedic implants to determine their potential allergic properties in vivo.

Generating an electrical signal by the electrode can be sustained or paroxysmal or stopped by a controller placed in the electrode or communicating with the electrode by wifi in order to ensure control of electrical current transfer to the desired place. The electrode according to the invention can also be used to stimulate striated muscle.

The present invention relates to a device which comprises one or more electrodes covered (or containing in their composition) with one or more layers of material inducing a piezoelectric effect in a system such as eukaryotic cells in cell culture, in a tissue such as the myocardium or in the contractile striated muscle. The device possibly also comprises electronic components such as a box for regulating the heart rate in the case of the myocardium, a module for

Processing data from the heart rate, a memory and elements for receiving and emitting instructions to the electrodes via WiFi or any other means of communication without physical connection to the electrodes to overcome the adverse effects of the muscle stimulation devices currently marketed. The electrodes of the invention contain a ferromagnetic element capable of interaction with the electromagnet of the steerable lead [symbol] S [symbol] .

A device according to the invention is characterized by the following description and this may not be considered as limiting the field of the invention.

In the case of a cardiac stimulation device (or “pacemaker”), the object of the device aims to control the stimulation/defibrillation (ie the creation of a controlled electrical impulse to induce a depolarization of the heart muscle).

The device according to the invention is composed of 2 distinct elements for the stimulation unit, which communicate with each other without physical connection, and an external programmer and a distinct element which is a steerable lead “S” used to facilitate extraction of the electrode to be changed or to be moved in another position during its implantation.

The first element A of the electrode is implanted in the heart (myocardium, endocardium or epicardium) is divided into four parts: the first part stimulates the surrounding environment (ie, create an electrical current that can induce cell depolarization). The second part collects the electrical myocardial potentials. The third part transmits collected data to the second element B and receives instructions from the second element B. The last part provides the energy required by the other parts of element A.

This latter part operates with a mixed source of energy with a conventional battery which is connected to the circuit and a second device using the piezoelectric effect which harvests part of the mechanical energy produced during contraction of the heart or kinetic energy produced during the movements of the person (the heart is moving at the same speed as the rest of the body) to charge the said conventional battery.

The second element B is outside the heart. It is made of six parts.

The first part receives data from the third portion of the first A. The second part will process the data received according to defined criteria. The third is used to store collected data according to defined criteria (memory). The fourth allows to issue instructions of stimulation to the first part of element A according to programming. The fifth part provides energy required by the second element. The sixth part contains the whole of the paradigms for adjustment of the second element (that is to say, the set of rules that defines an adapted stimulation according to electrical activity of the targeted tissue).

There is an external programmer to program the second element B.

The advantage of this device is to make easier the positioning of an electrode comprising a ferromagnetic element and to avoid the connecting stimulation lead which presently induces communication between the pacemaker/defibrillator and the heart: it reduces the risk of local complications (hematoma, infection, lead fracture, lead insulation dysfunction . . . ) and facilitates the implantation and replacement of pacemakers since firstly the element B is not necessarily located near a vascular structure which eliminates the risk of subclavian vein thrombosis and secondly, eliminates the risk of damage to the leads during dissection when the pacemaker is replaced.

The advantage of this device is also able to increase the life of existing devices due to active participation of the electrode to produce energy.

This also reduces the volume of each element which leads to an aesthetic gain and comfort for the patient.

Finally, we can also think that in the case of patients with endocarditis linked to pacemaker it will not be necessary to remove all the device since the infection of the heart does not spread from the controlling box (or element B) to the electrode (currently the common attitude is the removal of all equipment, box and probe, which is dangerous when the leads are old and adherent to the vessels so that extraction of these leads involves a risk of serious vascular damages which can lead sometimes to heavy heart surgery). In the present invention risks are limited and controlled by using the effects of the magnetic field induced by the lead “S” near the electrode having the ferromagnetic element when said extraction is performed.

Another option is to leave actual leads and to introduce additional leads which increases the risk of infection.

Finally it is likely that the risk of endocarditis is decreased with the device of the invention as the total volume of material in the heart will be lower and re-endothelialization of the whole implanted device will be possible so that an infection graft is less likely to occur.

In a preferred embodiment of the invention, the stimulation electrode is formed in part by a ferromagnetic metal. In this latter case, another material independent of the electrode is defined and allows the mobilization of said implanted electrode according to the invention.

This equipment is designed as an electromagnet positioned at the end of a steerable electrophysiology lead (S).

This lead “S” is similar to commercially available other steerable electrophysiology lead as far as biocompatibility is concerned. We can therefore bring this lead S near the implanted electrode. Then the electromagnet is activated. As a consequence, the electrode and the lead S are fixed together. We can therefore move the electrode under the control of an operator by acting on the displacement of the lead S.

In this way, the implantation/extraction rod (19) can be repositioned in the cavity (15) located at the center of the electrode and we can perform the inverse operation performed during implantation and thereby remove the electrode.

The electromagnet is designed according to the desired power in order to move the variant electrode which comprises a ferromagnetic element) according to the invention.

The contact force between the two objects (the variant electrode and the lead “S”), is related to the contact surface between the variant electrode and the lead S and the strength of the magnetic field generated by the electromagnet.

For example, a contact surface of 5 sq.cm and a magnetic field of 1 Tesla, induce a contact force of 205 Newtons which is equivalent to a weight of 21 kg in our gravity.

In the field of the invention as a preferred example of the electromagnet, it will be selected to induce a contact force between 30 to 100 Newtons.

As an indication, a summary of the current technique of implanting a pacemaker is described below. It comprises the following steps:
1—incision near the deltopectoral groove as close to a venous vascular structure (cephalic vein, axillary vein or subclavian vein),
2—dissection to the fascia of the pectoralis major.
3—Making the lodge of the pacemaker below the fascia of the pectoralis major.
4—venous access (cephalic vein/subclavian vein . . . ) according to Seldinger's technique.
5—fixation of the atrial/ventricular leads into the selected heart chamber with screw or tines in the myocardium of the atrium or the ventricle.
6—Fixation of each lead at their proximal end by a non-absorbable thread.
7—connection between the lead and the stimulator.
8. pacemaker implantation in the lodge in Step 3.
9. closing layer by layer of the skin.
10. programming the pacemaker

The proposed use of the device according to the invention technique is identical to steps 1 to 4. Steps 5 and 6 are deleted when the connection between the electrode A and the element B is achieved with WiFi.

To remove the variant electrode according to the invention, the said electrode comprising a ferromagnetic material, the physician uses a steerable lead S according to the invention. After introduction into a vascular structure, the lead S is led near the fixed electrode. The lead “S” is positioned near the electrode of the invention containing the ferromagnetic material. An electrical current is applied to the lead “S” which generates a magnetic field. This magnetic field allows to move the electrode from its original location in the amorphous tissue of the patient and to clear up the electrode's hollow in which will be brought the extraction rod to achieve the extraction of the electrode through a vascular structure and without surgical intervention.

The device according to the invention improves prior art since on the one's hand the implanted device containing the second element B is smaller which provides a technical and aesthetic advantage, and on the other hand, since the absence of lead(s) avoids the complications inherent to them (fractures, insulation breaks . . . ) and facilitates the change of the second element B. Finally, the lack of connection between the element A (where the electrode is according to the invention or variant electrode) and the element B makes easier the positioning of the first element A at the most suitable place without the need to be close to a vascular access.

The proposed herein improvement also reflects the self-powered mode of the element A which can charge a conventional battery (in part or fully) by the harvested energy produced by myocardial contraction using the piezoelectric effect. Indeed, at the time of myocardial contraction, there is an increase of intracardiac pressure which can be used by a device using the piezoelectric effect. This allows an increased duration of the battery of the element B which has not to supply electrical power to element A. This enhancement applies to the variant electrode according to the invention which will be then partly made with a piezoelectric element.

In a preferred embodiment according to the invention, the elements A and B in FIG. 1-A, are absolutely not linked physically. The electrode constituting the element A is implanted in the myocardium without any link with the element B (box). The effects of stimulation are managed with a wireless system, WiFi for example. In this preferred embodiment, the box size is much smaller than that used in pacemakers of the prior art, because it doesn't contain anymore space allocated to provide energy to the electrode(s), the latter having its separate specific source of energy.

The electrode or electrodes or variant electrodes according to the invention, which comprise a ferromagnetic material, can produce enough energy to operate when it is necessary to stimulate the myocardium. They can also, as another variant, produce and store energy generated by the said piezoelectric effect for later use, for example, stimulate the myocardium. A variant of the system is the presence in the electrode of a battery that can be charged by the piezoelectric electricity possibly via a capacitor.

The invention also relates to a fixation means of the electrode according to the invention, on the muscle to stimulate (eg the heart muscle). The electrodes are led to the implantation area in the heart by the venous or arterial system. For example, it can be used as a passageway, the femoral vein or subclavian vein sub or jugular vein or femoral artery in which will be moved the electrode having an attachment mean in the myocardium. The electrode is conducted in a sheath and mobilized on a guide or an steerable lead with or without an electromagnet for example, by a conventional surgical technique known to those skilled in the art, to the implantation area.

The electrode will be released from the steerable lead according to the invention or from any guide or from the most appropriate mean to mobilize the electrode until it is attached to heart. An electrode according to the invention is shown as an example in FIG. 3. This representation is not exhaustive.

To illustrate the present invention, the figures attached to this description are representations of achievement of different aspects of the invention. These different representations are not exhaustive.

FIG. 1: Element A is formed by an electrode composed partly or entirely by a material with a piezo-electric effect and in part by a ferromagnetic material with a control circuit (2) having means for emission and reception of electrical signal and a stimulation module (3), which stimulates a muscle or a nerve cell or any cell that can be excited (6) by an electric current and optionally a portion designed for energy storage (eg rechargeable battery) (4). The latter is powered in part or totally by the piezoelectricity. Element A is connected with element B by means of wireless communication, for example by WiFi. There is a module for detecting the electrical activity (spontaneous or induced) (5). The piezoelectric metal (1) undergoes mechanical stresses (due to movements from cardiac contraction or walking) which are converted into electrical energy and stored in the rechargeable battery (4).

Element B is constituted by an energy storage space such as a battery (7) and also includes emission (8)/reception (9) means of electrical signals from or destined to Element A. It contains the electronic components (10) for programming element B with the help of an external programmer (11). Element B contains also a memory and an electronic brain for processing information. These elements will serve as a model for the operations of the electrode.

FIG. 2: Representation in a longitudinal section of an electrode according to the invention. It has an empty space in its center (15) limited in part with a layer having a piezoelectric effect (16.1) and in part by ferromagnetic material (16.2) the said layer is itself optionally coated with a thin film (17) chosen to avoid the reactions of platelet aggregation or thrombosis or allergic reactions of the organism in the presence of foreign compositions.

FIG. 3: representation in a longitudinal section of the portion of the electrode in which there is a screw-type means (18) or equivalent that is used to fix and maintain the electrode in place in the myocardium. The screw can be made of a material containing a ferromagnetic portion as a variant. The ferromagnetic material (33) is only present in the electrode variant of the invention.

In the inner central portion of the electrode (15) is an extraction/implantation rod (19) which can be fixed temporarily to the inner wall of the electrode (20). A guide wire (21) leads the implantation and extraction rod in the hollow of the electrode. This is the association with the extraction rod/guide wire and electrode (or electrode variant according to the invention) which is brought to the site of implantation of the electrode with or without using the lead “S” according the nature of the electrode (with or without a ferromagnetic material). When the electrode is fixed, the whole device (extraction rod/guide wire) is separated from the electrode and is removed from the vascular axis, for example the vein in which it has been conveyed. If the electrode is to be subsequently removed from the myocardium, the reverse operation is performed: thus, the extraction rod and the guide wire are put into the hollow of the electrode by means of a catheter or a guide wire with or without the help of the steerable lead “S” which contains the electromagnet. The extraction rod/guide will be fixed to the inner portion of the electrode for example, allowing to stop or move the electrode or the embodied electrode of the invention. As a consequence of it, it will be easier to enter the top of the extraction rod in the recess (15) with the help of the magnetic field emitted by the lead “S”. The magnetic field gives the physician freedom to move or freeze the electrode according to the intervention's needs and in particular to extract the electrode with the rod with which it has first been properly positioned. The lead S will be brought outside together with the electrode, passing through the most appropriate vascular path. There is an element (29) which can induce a depolarization of muscular cells. This element is in contact with a battery (30) which is rechargeable with piezoelectricity. This method of implantation/explantation is not exhaustive. There is also a module for detecting electrical impulses (31). This latter module and the stimulation unit (29) are controlled by a control unit (32) which receives instructions from element B (see FIG. 1) for triggering the stimulation or not.

FIG. 4: Comparison between a “classical” pacemaker (1) and a pacemaker according to the invention (2) containing a pacemaker including a ferromagnetic material in the case where the electrode is a variant electrode according to the embodiment of the invention. Implantations areas of the electrode are noted as an example.
(1) [symbol] classical [symbol] pacemaker in which the electrode (22) and the element B (23) are linked by extensions (leads) (24). These extensions are for example electrical housed wires. They are necessary for obtaining stimulation activity (25) on the control of element B. The element B comprises a battery in order to stimulate the myocardium, to power the electrode, the memory and program modules.
(2): the pacemaker according to the invention: the electrode (26) is covered with a piezoelectric component which under the effect of stress of the heart or the kinetic energy transmitted to the heart (28) will generate the electricity that is stored in the electrode or the variant electrode according to the invention which also contains a ferromagnetic material. As stresses are permanent, energy storage may be low. This reduces the volume of the electrode. The element B (27) is not connected to the electrode as it does not need to supply energy in the electrode. It has a battery for its own operation. It communicates with the electrode via a wireless connection (WiFi for example).

The device according to the invention relates to a medical device and its individual components comprising:
An electrode with a piezoelectric effect, all or part of the electrode being at least partly made with a material inducing a piezoelectric effect. Optionally, as an alternative, the electrode's material also contains a ferromagnetic material which can be magnetized in a magnetic field induced by the electromagnet of the steerable lead “S” according to the invention. The ferromagnetic material (such as iron) is not itself spontaneously a generator of a magnetic field.
A steerable lead “S” whose diameter is compatible with its way through a vascular axis that contains an electromagnet (made for example by Mecalectro, France) and is covered by a thin film compatible with its use in allergic subjects or with anti-platelets properties or anti anti-allergic properties.
An electrode operating wirelessly with the element B (with WiFi for example) for the transmission of data to the element B and for the reception of data from the element B.
A pacemaker implanting set comprising the said electrode according to the invention, implantation and explantation means of the electrode without operating directly on the element B or removing it when the electrode is changed or a new electrode is implanted.

A variant of the invention allows to reduce the volume of the box in the pacemakers prior art: it is permitted by replacing the electrodes linked to the box by a classical electrical wire, with electrodes linked to the box with electrical wire which, according to the materials in their composition, have the ability to be active without addition of energy from the box. The reduction in the volume of the box saves space during implantation through the use of electrodes according to the invention that do not require additional energy from the pacemaker box.

The invention also relates to a method for producing a means of muscle stimulation such as the heart muscle characterized by an electrode which can produce energy from the piezoelectric effect and means to fix the electrode.

The method according to the invention also comprises the programming of the pacemaker case without direct contact with the electrode and the monitoring, as appropriate, of the pacemaker's activity, such as recording of pacing graphics, electrical activities and any data useful for the pacemaker monitoring over a specified period.

The invention relates to a system of bio-stimulation, including a bio-stimulator configured for, on the one's hand, implanting an electrode according to the invention which can create enough electrical power to maintain the electrode activated and, on the other hand ,for the collection and data processing related to the myocardial stimulation. The latter is possible with the help of an energy generator according to the invention.

The invention also relates to a method of measuring the minimum energy required to induce an adapted electrical activity taking into account the adjustments.

The invention relates to a muscle stimulation device characterized in that it comprises at least one electrode formed in whole or in part by a material generating a piezoelectric effect and configured for bi-directional communication with an element positioned outside of the electrode's implantation site. A variant of said electrode contains a ferromagnetic material.

The device according to the invention comprises a muscle stimulator, in particular a cardiac muscular stimulator.

The pacemaker includes electrodes according to the invention, whether or not connected by electrical wires to one of the parts constituting the box (element B) of the pacemaker.

The device according to the invention contains at least two separate elements (A and B): at least one electrode (element A) that works with an electrical energy supplied by the piezoelectric effect due to the nature of the composition of materials of said electrodes, and a box (element B) supplied with a source of non-renewable energy. Said device operating with or without a lead inside the patient's body. As an embodiment, a variant electrode comprises a ferromagnetic material.

The device of the invention includes electrodes and variant electrodes that may have in their center a hollow portion, attachment means to the myocardium and optionally a guide and optionally a steerable rod, these last two objects being retractable and allow the implantation or extraction of said electrode. A variant of the invention can be the implantation or extraction of the electrode by the use of magnetism: the electrode being formed partly or totally with a magnetic material, it is possible, by positioning an electromagnet near the electrode to fix the latter and therefore making possible to extract it. The implantation of the electrode may also be performed by using magnetism by deploying the means of fixation of the electrode to the cell or organ to be stimulated, for example the myocardium.

The present invention relates to a pacemaker system comprising an electrode or a variant electrode, according to the invention, implanted in the myocardium and configured to be powered with the in situ generated energy independently of the energy produced by the box (element B) for the specific needs of it.

The invention also relates to a method of controlling electrical pulses in a muscle tissue characterized by measuring the endocardial electrical potentials and the frequency of planned or spontaneous impulses transmitted to the can of the stimulator. This method makes the electrodes work independently from the box energy.

The invention relates to an electrode formed totally or partly with materials that create a piezoelectric effect as a primary or secondary source of energy to ensure the activity of muscular stimulation according to a defined pattern, the collection of intracardiac electrical potentials stimulated or spontaneous and the communication with the box (element B). The said variant electrode also contains a ferromagnetic material.

The invention relates to a method of using the device according to the invention, characterized by measuring the effect of stimulation on the myocardium or the measurement of the spontaneous electrical activity of the myocardium and the comparison with the programming of the element B according to the instructions of the physician. The result of this comparison is the activation or inhibition of electrical stimulation by the electrode.

The invention also relates to a method of implantation and extraction of an electrode or a variant electrode according to the invention: it uses a steerable lead “S” emitting a magnetic field on demand, said method characterized by the use of properties of a ferromagnetic material present on or within the electrode and of an electromagnet on the surface of or in the lead “S”. These properties allow movements of variant electrode under control of the physician for its adequate positioning in the heart as well as for its extraction.

Examples of different options for carrying out the invention are illustrative of the invention and are not to be considered limited by them. In contrast, the present invention includes all variants implementing innovative features built on the functional equivalents.

1-10. (canceled)

11. An implantable muscular or nervous stimulation electrode generating and transmitting autonomously an electrical signal to a muscle or to a nervous tissue or a nerve in the human or animal body, wherein the electrode is constituted totally or partly by one or several piezoelectric materials, located on the whole surface of the electrode or close to the surface alternatively with one or more components present in the said electrode, generating by a piezoelectric effect produced by a physical stress exerted on the electrode a source of energy.

12. An electrode according to claim 11 wherein the material or the materials generating a piezoelectric effect are chosen among: ferroelectric materials with a perovskite structure, LZT (lead zirconate titanate), BaTiO3 (barium titanate) or KNbO3 (potassium niobate), the derivatives thereof, MEMS (micro-electro-mechanical systems), semiconductors from the group III-V “zinc blende” and group II-VI with a Wurtzite structure, or Quartz.

13. An electrode according to claim 11 wherein said electrode is associated with a battery allowing to ensure i) the muscular or nervous stimulation activity and ii) the records of stimulated or spontaneous electric potentials, said electrode comprising a ferromagnetic material and being coated with a thin film for preventing platelet aggregation reaction or allergic reactions and being capable of communicating with a separate distinct element without physical connection.

14. An electrode according to claim 11 wherein said electrode is totally extractable.

15. An electrode according to claim 11 comprising a material with piezoelectric effect and a ferromagnetic material.

16. An electrode according to claim 11 wherein the ferromagnetic material is not a source of electrical energy.

17. An electrode according to claim 11 is coated with a thin film for preventing platelet aggregation reaction or thrombosis or allergic reactions and it comprises a cavity.

18. An electrode according to claim 11 comprising a rechargeable battery through the piezoelectric effect, a stimulation module for inducing a depolarization of muscle cells or nerves, a detection module of electrical impulses, and a control module.

19. An implantable muscle stimulation device comprising at least two separated distinct elements:
at least one electrode according to claim 11 i) constituted totally or partly by one or several materials that produce a piezoelectric effect and ii) configured for bidirectional communication with
a box having electronic modules necessary for the storage, processing and bidirectional communication of signals with the at least one electrode and/or with an external programmer

20. An implantable muscle stimulation device according to claim 19 wherein i) the electrode is constituted with one or more materials generating a piezoelectric effect from a physical stress exerted on the electrode and wherein the box contains i) a stockage module collecting the information originating from the electrode and ii) a dedicated module for analyzing them and iii) a battery and iv) a communication module acting with an outside programmer.

21. An implantable muscle stimulation device according to claim 19 comprising the electrode and a steerable electrophysiology lead having an electromagnet powered outside of the patient and activable on request.

22. A device according to claim 19 wherein the electrode comprises a ferromagnetic element.

23. A device according to claim 19 further comprising a steerable lead containing an electromagnet capable to interact with the electrode containing a ferromagnetic material when close to the said electromagnet of the lead and ii) the stimulator is a pacemaker.

24. A device according to claim 19 further comprising a stimulator's housing box and the at least one electrode containing a ferromagnetic material capable to interact with a movable electromagnet said electromagnet being powered by an external electric current distinct of the said device.

25. A device according to claims 19 wherein the stimulator's box and the at least one electrode comprising a piezoelectric effect material and a ferromagnetic material or optionally a non-magnetic magnetizable material, are communicating by a wireless communication such as WiFi or by insulated electrical wires.

26. A method of treatment of a patient affected with heart conditions comprising the steps of:
a) implantation of a muscle stimulation device according to claim 19,
b) measurement and control of the energy produced by the electrode and stored,
c) providing selectively and appropriately the quantity of stored stimulation energy to the heart of a patient and optionally,
d) extracting the implanted electrode i) by fixation of said electrode by electromagnetic interactions with the steerable electrophysiology lead having an activated electromagnet ii) by moving through the blood vessels all necessary surgery elements including the rod close to the location of the electrode and iii) by allowing the total extraction of the electrode out of the body of the patient and optionally
e) replacing the extracted electrode by a new one at an appropriate location selected by the physician on or inside the heart.

27. A method according claim 26, wherein during the extraction step an extraction device is used for extracting the implanted electrode according to step d) the said device being constituted i) by the electrode containing a ferromagnetic element not involved in the energy production nor its storage and ii) by said the steerable electrophysiology lead having an electromagnet activated as magnetized on request which make a favorable close spatial arrangement complex with said electrode.
